(19) United States
(12) Patent Application Publication
(10) Pub. No.: US 2018/0148390 A1
(43) Pub. Date: May 31, 2018

(54) METHOD AND COMPOSITION FOR RETAINING NUTRIENTS IN SOIL AT PLANTING SITES

(71) Applicant: WaterScience, Inc., Peoria, IL (US)

(72) Inventor: Robert J. Littmann, Peoria, IL (US)

(73) Assignee: WaterScience, Inc., Peoria, IL (US)

(21) Appl. No.: 15/793,533

(22) Filed: Oct. 25, 2017

Related U.S. Application Data
(60) Provisional application No. 62/412,560, filed on Oct. 25, 2016.
(60) Provisional application No. 62/412,548, filed on Oct. 25, 2016.

(51) Int. Cl. C05F 11/00; C07C 229/08; C07C 237/06; C07C 229/24; C07D 233/64; C07C 229/26; C07C 229/36; C07D 209/20; C07C 323/25; C07C 279/12

(52) U.S. Cl. CPC C05F 11/00; C07C 229/08; C07C 237/06; C07C 229/24; C07C 279/12; C07C 229/26; C07C 229/36; C07D 209/20; C07C 323/25; C07D 233/64

(57) ABSTRACT

Methods and compositions of retaining nutrients in soil at planting sites is disclosed herein. In some embodiments, the composition includes at least one modified amino acid. The at least one modified amino acid is modified by at least one of protonation, ammonia modification, or guanidine modification. In some embodiments, the composition further includes at least on unmodified amino acid.

### CROSS REFERENCE TO RELATED APPLICATIONS

This application claims the benefit under 35 U.S.C. § 119 (e) of U.S. Provisional Application No. 62/412,548, filed Oct. 25, 2016, and U.S. Provisional Application No. 62/412,560, filed Oct. 25, 2016, the contents of which are incorporated herein by reference in their entirety.

### FIELD

Compositions and methods for retaining nutrients in soil at planting sites are provided. Methods of making components of compositions, such as amino acids, are also provided.

### BACKGROUND

Farmers striving for high crop yields use excessive amounts of nutrient-providing fertilizers, such as natural (manures) and chemically synthesized fertilizers. Most companies and individuals with lawns, gardens, golf courses, etc., want them to look green, fruitful and vibrant use excessive amounts of fertilizers. Nutrient run-off and ground water contamination may be caused by excessive nutrient application to agricultural lands, golf courses, parks, nurseries, gardens, lawns, and other sites. The run-off of nutrients causes hypoxia which may cause the death and growth inhibition of most aquatic life. Vivid examples are the dead zones in the Gulf of Mexico, Chesapeake Bay and Lake Erie. Surface water and ground water contamination with nutrients may cause increased potable water treatment costs, and expensive, complicated processes.

Plants require 16 nutrients to grow. Non-mineral nutrients include hydrogen, oxygen and carbon. These nutrients are found in the air and water. Plants use energy from the sun to change carbon dioxide and water into starches and sugars through photosynthesis. These starches are the plant's food. Since plants get carbon, hydrogen and oxygen from the air and water there is little farmers can do (other than locate plants in sunny areas/irrigate when rainfall is low) to control how much of these nutrients are available to the plants.

The 13 mineral nutrients, which come from the soil, are dissolved in water and adsorbed through a plant's roots. There are not always enough of these nutrients in the soil for healthy plant growth. This is why farmers use fertilizers to add the nutrients to the soil. The mineral nutrients are divided into two groups: macronutrients and micronutrients.

Macronutrients can be broken into two more groups: primary and secondary nutrients. The primary nutrients are nitrogen, phosphorus, and potassium. These major nutrients usually are lacking from the soil because plants use large amounts for their growth and survival. The secondary nutrients are calcium, magnesium, and sulfur. There are usually enough of these nutrients in the soil, so fertilization with secondary nutrients is not always needed.

The 7 micronutrients are those elements essential for plant growth which are needed in only very small quantities. These elements are boron, copper, iron, chloride, manganese, molybdenum and zinc. If required micronutrients are usually available in the soil, and, in most cases, no supplemental addition is required.

Soils vary widely in composition, structure, and nutrient supply. Especially important from the nutritional perspective are inorganic and organic soil particles called colloids. Soil colloids retain nutrients for release into the soil solution where they are available for uptake by the roots. Soil colloids serve to maintain a reservoir of soluble nutrients.

The function of the colloidal soil fraction depends on two factors: (1) colloids present a large specific surface area, and (2) the colloidal surfaces carry a large number of charges. The charged surfaces in turn reversibly bind large numbers of ions, especially positively charged cations from the soil solution. This ability to retain and exchange cations on colloidal surfaces is the single most important property of soils, insofar as plant nutrition is concerned.

Colloidal clays supply predominately negative charges by virtue of the alumina and silica at the edges of the clay particle. Because colloidal carbon is derived largely from lignin and carbohydrates, it also carries negative charges arising from exposed carboxyl and hydroxyl groups.

Soil colloids are predominantly nonionic and anionically charged and, consequently, they do not tend to attract negatively-charged anions (in other words, the anion exchange capacity of soil colloids is relatively low). The result is that anions are not held in the soil but tend to be readily leached out by percolating ground water. This situation has important consequences for agricultural practice. Nutrients supplied in the form of anions must be provided in large quantities to ensure sufficient uptake by the plants. As a rule, farmers often find they must apply at least twice—sometimes more—the amount of nitrogen required for producing a crop.

Unfortunately much of the excess nitrate is leached into the ground water, and eventually finds its way into wells or into streams and lakes, where it contributes to problems of eutrophication by stimulating the growth of algae. Similar issues relate to the inefficient uptake of negatively charged phosphates (PO₄³⁻) and sulfate (SO₄²⁻) by plants, with subsequent problems resulting from nutrient runoff.

Plants vary on how much macronutrient (nitrogen, phosphorous, potassium) they require for robust growth. For example, corn requires high levels of nitrogen while legumes do not require any nitrogen as they are able to fix nitrogen requirements from the air.

There are three fundamental ways plants uptake nutrients through the root: 1) simple diffusion, occurs when a non-polar molecule, such as O₂, CO₂, and NH₃ follows a concentration gradient, moving passively through the cell lipid bilayer membrane without the use of transport proteins, 2) facilitated diffusion is the rapid movement of solutes or ions following a concentration gradient, facilitated by transport proteins; 3) Active transport is the uptake by cells of ions or molecules against a concentration gradient. This requires an energy source, usually ATP, to power molecular pumps that move the ions or molecules through the membrane.

Three of the important macronutrients, nitrogen, phosphorous and sulfur enter the plant cell wall in the form of anions. If these macronutrients are not retained in the soil in proper concentration to facilitate their transport across the plant cell wall, excess fertilization will be required to obtain optimum crop yields.

Methods being considered to control nutrient run-off include collection of run-off water and removal of nutrients. This increases pollution abatement capital and operating costs, and does not address ground water contamination or optimization of crop yields.

Another method being considered is to grow scavenger plants around the perimeter of agricultural fields to capture the excess nutrients. This does not address the wastage of fertilizer usage nor does it address ground water contamination or the desirability of increased crop yield.

Increase of the soil Cation Exchange Capacity (CEC) by usage of humic acid is neither efficient nor effective at addressing the need for retaining anions. The theory of increasing CEC is that ammonium, while being a cation readily nitrifies to nitrates which are anions and thus not retained in the solid. Nitrification blockers are an additional expense and only partially effective. Frequently nitrogen is applied in the form of ammonium nitrate. The nitrate form of nitrogen is negatively charged and not affected by CEC. Further, phosphates and sulfates are also anions and not effectively retained by CEC.

Clays, which are the main source of CEC, have low efficiency, being less than 10% as efficient in retaining cations. Many cations in the soil which are needed by plants are actually anion complexes and thus are not retained by CEC. Moreover, clays are weakly charged. As such, there is minimal inhibition of hydraulic leaching of bound cations during irrigation or rains.

CEC, as traditionally determined by standard soil analyses, does not address the lack of specificity or selectivity of complexing and retaining cations in soils. Lack of specificity or selectivity of ion exchange sites in soil requires overly large dosages of CEC in soil. For example if 200 pounds of nitrogen as ammonium based fertilizer is applied to the top twelve inches of soil, the milliequivalents of Ammonia as N per 100 grams applied to the soil is 0.357. The ammonium cation is retained in the soil by ion exchange methods. As an ion held in an ion exchange complex it is essentially replaced by all other cations in the soil. To overcome the lack of selectivity or specificity of the smaller, important ions in soil, the soil CEC must be increased by 28 fold or more. Typically, desired CEC in fertile soil is targeted to exceed 10 meq/100 grams.

Anion Exchange Capacity (AEC) as traditionally determined by standard soil analyses do not address the lack of specificity or selectivity of complexing and retaining anions in soils. Traditionally, AEC when it is determined (rarely) merely measures whether phosphate forms a complex with soil after the soil has been pretreated with a calcium salt in the test column. Therefore, traditionally AEC is a go/no go test for whether calcium treated soil reacts with phosphate at the pH of the test condition selected. Thus, there is no delineation of the various forms of phosphorous in soil nor is there indication of the selectivity or specificity of soil for other important anions requiring complexing and retention in soils including organically bound and inorganically bound nitrite, nitrate, and various forms of sulfur and organically bound phosphorous.

Almost all soils have low or no AEC. Any AEC in soil has no selectivity or specificity for one anion over another but rather follows the physical laws of ion exchange as referenced above for CEC.

Many farmers, in their quest to lower their operating costs, apply biosolids generated from the treatment of waste water by municipal or private wastewater treatment plants. When farmers apply biosolids to their farms they are trying to obtain free nutrients contained in the biosolids.

There are several problems with the application of biosolids to agricultural and non-agricultural sites. These problems include: 1) biosolids contain toxic and hazardous metals; 2) biosolids contain pathogens; 3) biosolids can attract and propagate vectors and in so doing can spread disease; 4) biosolids contain PCP and Ps (Personal Care Products and Pharmaceuticals) and other toxic and hazardous organics which should not be allowed to accumulate and concentrate in the food chain.

Biosolids comprise strong anionic charges. When biosolids are added to soil, along with the strongly anionically charged particles, non-selective cation exchange capacity is added to the soil but most importantly, along with the non-selective CEC, negative charges are added in high concentration. These negative charges repulse anions in the soil to runoff, loss to tile drainage water or loss by percolation through the ground, contaminating ground water.

Loss of anions from agricultural and non-agricultural sites seems to occur through coulombic forces causing the highly negatively charged organics to disperse anion nutrients from soil sites through negative charge repulsion forces, thus exacerbating pollution of drinking water supplies and important waterways.

Current municipal and private wastewater treatment methods are designed and operated to remove mostly biochemically degradable solids from the treated wastewater. Non-biodegradable solids removal is limited to physical methods, as such soluble, non-biodegradable solids are minimally removed from wastewater.

Current municipal and private wastewater treatment methods are designed and operated to minimize the mass or volume of solid residue remaining after wastewater treatment. One widely used method of solid residue reduction is anaerobic digestion. In the process of anaerobic digestion, about 40 wt % to 50 wt % of the volatile solids in the solid residue are biodegraded. While anaerobic digestion reduces wastewater treatment solid residues, it also consumes valuable nutrients which can be beneficially and safely reused on agricultural and non-agricultural sites.

There is a need in the art for controlling loss of nutrients from planting sites, and for recovering nutrients from waste treatment, instead of using harmful biosolids.

### BRIEF SUMMARY OF INVENTION

The present invention includes compositions having a specific ion complexing agent to retain nutrients in soil at planting sites, methods of making the same, and methods of retaining nutrients in soil using the same. The specific ion complexing agent includes at least one modified amino acid, where the modification improves the retention of the amino acid in soil or the ability of the amino acid to retain nutrients. For example, one modification is protonation of the amino acid, which improves retention of the amino acid in negatively charged soil.

The method of making can further include using waste water as a nutrient source in a bioreactor to facilitate the formation of amino acids. As a result, the amount of biosolids generated during waste water treatment can be reduced. Moreover, the amino acids produced can be further utilized to remove additional nutrients from the waste water.

In some embodiments, a composition includes at least one modified amino acid.

In some embodiments, the at least one modified amino acid is selected from the group consisting of a protonated amino acid, an ammonia modified amino acid, a guanidine functionalized amino acid, and mixtures thereof.

In some embodiments, the composition further includes an unmodified amino acid.

In some embodiments, the unmodified amino acid is selected from the group consisting of arginine, lysine, and histidine.

In some embodiments, the composition includes histidine, protonated alanine, lysine, and protonated phenylalanine.

In some embodiments, the composition includes histidine, ammonia modified glutamic acid, ammonia modified valine, ammonia modified tryptophan, and ammonia modified methionine.

In some embodiments, the composition includes guanidine modified leucine, guanidine modified isoleucine, guanidine modified asparagine, and guanidine modified valine.

In some embodiments, the at least one unmodified amino acid is selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

In some embodiments, the at least one unmodified amino acid is selected from the group consisting of a-amino-n-butyric acid, norvaline, norleucine, alloisoleucine, t-leucine, a-amino-n-heptanoic acid, proline, pipecolic acid, a, β-diaminopropionic acid, a, γ-diaminobutyric acid, ornithine, allothreonine, homocysteine, homoserine, B-alanine, B-amino-n-butyric acid, B-aminoisobutyric acid, isovaline, sarcosine, N-ethyl glycine, N-propyl glycine, N-isopropyl glycine, N-methyl β-alanine, N-ethyl β-alanine, N-methyl alanine, N-ethyl alanine, isoserine, a-hydroxy-γ-aminobutyric acid, and mixtures thereof.

In some embodiments, the protonated amino acid is a protonated form of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

In some embodiments, the ammonia modified amino acid is an ammonia modified form of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

In some embodiments, the composition includes guanidine modified amino acid is a guanidine modified form of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

In some embodiments, the protonated amino acid is a protonated form of a-amino-n-butyric acid, norvaline, norleucine, alloisoleucine, t-leucine, a-amino-n-heptanoic acid, proline, pipecolic acid, a, β-diaminopropionic acid, a, γ-diaminobutyric acid, ornithine, allothreonine, homocysteine, homoserine, B-alanine, B-amino-n-butyric acid, B-aminoisobutyric acid, isovaline, sarcosine, N-ethyl glycine, N-propyl glycine, N-isopropyl glycine, N-methyl β-alanine, N-ethyl β-alanine, N-methyl alanine, N-ethyl alanine, isoserine, a-hydroxy-γ-aminobutyric acid, and mixtures thereof.

In some embodiments, the ammonia modified amino acid is an ammonia modified form of a-amino-n-butyric acid, norvaline, norleucine, alloisoleucine, t-leucine, a-amino-n-heptanoic acid, proline, pipecolic acid, a, β-diaminopropionic acid, a, γ-diaminobutyric acid, ornithine, allothreonine, homocysteine, homoserine, B-alanine, B-amino-n-butyric acid, B-aminoisobutyric acid, isovaline, sarcosine, N-ethyl glycine, N-propyl glycine, N-isopropyl glycine, N-methyl β-alanine, N-ethyl β-alanine, N-methyl alanine, N-ethyl alanine, isoserine, α-hydroxy-γ-aminobutyric acid, and mixtures thereof.

In some embodiments, a method of making a modified amino acid includes providing nutrients to a bioreactor, where the bioreactor includes a microorganism capable of utilizing the nutrients to manufacture an amino acid. In some embodiments, the amino acid manufactured is an unmodified amino acid.

In some embodiments, the unmodified amino acid is reacted to form a protonated amino acid, an ammonia modified amino acid, or a guanidine modified amino acid.

In some embodiments, a method of waste water treatment includes diverting a waste sludge to the bioreactor, wherein the waste sludge includes nutrients capable of being utilized by the microorganisms to manufacture an amino acid. In some embodiments, the amino acid is reacted to form a modified amino acid. In some embodiments, the modified amino acid is provided to the waste water stream to remove nutrients therefrom.

In some embodiments, a method of retaining nutrients in soil at a planting site includes provide the composition to the soil, wherein the composition selectively binds nutrients in the soil.

### BRIEF DESCRIPTION OF DRAWINGS

FIG. 1 depicts a process flow diagram of waste water treatment plant (WWTP).

FIG. 2 depicts a process flow diagram of the WWTP of FIG. 1 modified in accordance with some embodiments of the present invention.

FIG. 3 depicts a process flow diagram for a biosynthesis process.

FIG. 4 depicts a schematic of a bioreactor.

### DETAILED DESCRIPTION

This inventor has discovered that amino acids are able to selectively complex and retain nutrients in agricultural soils. Most amino acids must first be modified to enable attachment to the high negative charges in soils. Amino acids are charged or non-charged. Negatively charged amino acids will be repulsed from the soil negative charges and lost during irrigation or rainfall. Non-charged amino acids will likewise be lost to runoff or ground water percolation during irrigation or rainfall. Of the 22 essential amino acids only arginine, histidine and lysine are positively charged; these three can bind with the negative charges in the soil and not be lost to runoff or ground water percolation. There are hundreds of non-essential acids which can be employed in this invention.

Composition

A composition for complexing and retaining nutrients is disclosed herein. The composition includes at least one modified amino acid. As used herein a ‘modified amino acid’ has the ability to be complexed and retained in the soil and be able to complex and retain available negatively and positively charged nutrients in the soil. Modifications to the amino acid may include protonation, ammonium addition, and/or guanine addition as discussed herein. The modified amino acids can retain available ionic nutrients that results from microbial degradation of soil organics and/or from addition of synthetic fertilizers. The ionic nutrients include anions and cations. At least some ionic nutrients require modification of an amino acid to be effectively retained in soils and to concurrently complex and retain other ionic nutrients.

Typical ionic nutrients include compounds that containing nitrogen, phosphorus, potassium, sulfur boron, alkaline earth and transition metals. Exemplary ionic nutrients can include nitrates, nitrites, sulfates, phosphates, ammonium, potassium, boron, calcium, magnesium, transition metals. All of the previously listed nutrients can be specifically complexed and retained including nitrates, nitrites, sulfates, phosphates, ammonium, potassium, boron, calcium, magnesium, transition metals. Ionic nutrients can be found in various sources of fertilizers, such as nitrogen, phosphorous and potassium and transition metals.

The composition may include at least one modified amino acid which is suitable for complexing and retaining ionic nutrients. In some embodiments, the composition may include more than one modified amino acid. In some embodiments, the composition may include a combination of unmodified amino acids and modified amino acids. As used herein, an ‘unmodified amino acid’ is an amino acid that has not be chemically altered to improve complexing with ionic nutrients. The amounts of a modified amino acid and/or an unmodified amino acid present in the composition can vary. In some embodiments, an amount of a modified amino acid can range from about 1 wt % to about 70 wt %. In some embodiments, an amount of a modified amino acid can range from about 5 wt % to about 60 wt %. In some embodiments, an amount of a modified amino acid can range from about 10 wt % to about 50 wt %. In some embodiments, an amount of a modified amino acid can range from about 5 wt % to about 10 wt %. In some embodiments, an amount of a modified amino acid can range from about 1 wt % to about 5 wt %. In some embodiments, an amount of a modified amino acid can range from about 70 wt % to about 90 wt %. A similar range of amounts can be used for the unmodified amino acid.

The composition can be tailored based on the crop being raised, the nutrients available in the soil, and the like. For example, it can be desirable to retain ionic nutrients containing nitrogen, phosphorus, potassium, and/or sulfur. These ionic nutrients may be present in the soil in various amounts or added to the soil by way of fertilizer in various amounts. For instance, in some embodiments, large amounts of an ionic nutrient including nitrogen may be present, and minor amounts of ionic nutrients including phosphorous, potassium, sulfur and cations may be present. Accordingly, a composition that includes modified amino acids can be tailored to include an amino acid that complexes the ionic nutrient including nitrate, an amino acid that complexes the ionic nutrient including phosphate, an amino acid that complexes the ionic nutrient including potassium, and an amino acid that complexes the ionic nutrient including sulfate, and an amino acid that complexes the ionic nutrient including cations. For example, these amino acids may be present in the composition in amounts of 70 weight percent (wt %), 10 wt %, 10 wt %, 5 wt %, and 5 wt %, respectively. These amounts can vary based on a crop being grown or based on a crop rotation pattern. The dosage of the composition can range from 100 to 12,000 pounds per acre (lb/acre). In some embodiments the dosage can depend upon prior application of the composition as carry over can occur year to year.

The composition may further comprise other ingredients, such as granulating agents and nucleating agents. Exemplary granulating agents can include vegetable oil, and other granulating agents known in the art. Exemplary nucleating agents can include potash. The other ingredients are present in the composition in an amount of about 10 wt % or less. In some embodiments, the amount of other ingredients in the composition can range from about 0.1 wt % to about 10 wt %. In some embodiments, the amount of other ingredients in the composition can range from about 1 wt % to about 10 wt %. In some embodiments, the amount of other ingredients in the composition can range from about 5 wt % to about 10 wt %. The composition can be a solid. In some embodiments, the solid has 10 wt % moisture or less.

One exemplary composition includes unmodified arginine present in an amount of up to about 70 wt %, unmodified histidine present in an amount of up to about 10 wt %, proton modified alanine present in an amount of up to about 10 wt %, lysine present in an amount of up to about 5 wt %, and proton modified phenylalanine present in an amount of up to about 5 wt %. The amino acids, modified or unmodified, can be present as a salt such as sulfate or hydrochloride in some embodiments. The composition can include other ingredients in an amount of up to about 10 wt %, such as granulating agents and/or nucleating agents. In this exemplary embodiment of the composition, arginine is beneficial in complexing and retaining nitrate and phosphate, lysine is beneficial in complexing and retaining nitrates and nitrites, histidine is beneficial in complexing and retaining sulfates.

One exemplary composition includes ammonia modified glutamic acid present in an amount of up to about 70 wt %, unmodified histidine present in an amount of up to 10 wt %, ammonia modified valine present in an amount of up to about 10 wt %, ammonia modified tryptophan present in an amount of up to about 5 wt %, and ammonia modified methionine present in an amount of up to about 5 wt %. The amino acids, modified or unmodified, can be present as a salt such as sulfate or hydrochloride in some embodiments. The composition can include other ingredients in an amount of up to about 10 wt %, such as granulating agents and/or nucleating agents. In some embodiments, the amino acids are present in their natural form and not as a chloride, dichloride or a salt. These forms can detrimentally increase the solubility of the amino acid in the soil.

One exemplary composition includes guanidine modified leucine present in an amount of up to about 70 wt %, guanidine modified isoleucine present in an amount of up to about 10 wt %, proton modified asparagine present in an amount of up to about 10 wt %, ammonia modified valine present in an amount of up to about 5 wt %, and ammonia modified alanine present in an amount of up to about 5 wt %. The amino acids, modified or unmodified, can be present as a salt such as sulfate or hydrochloride in some embodiments. The composition can include other ingredients in an amount of up to about 10 wt %, such as granulating agents and/or nucleating agents.

The composition improves complexing and retaining capability of amino acids for nutrient retention. The composition directly provides plants with nutrients which occurs when modifications add more nutrients which can be directly assimilated by the plant roots via the intact amino acid. Thus the more nitrogen, phosphorous and potassium the amino acid contains, the less stressful it is for plants to acquire these nutrients in individual form, i.e., such as in the form of potassium, nitrates, ammonium or phosphate.

Modified Amino Acids

Modified Amino Acids are described herein. One of ordinary skill in the art will understand that the modified amino acids provided herein is not an exhaustive list, and the modification processes disclosed herein can be utilized to modify other amino acids not discussed herein to provide similar benefits.

Protonated amine functional groups having a positive charge can complex and retain anions in soil, such as nitrates, nitrites, sulfates, and phosphates. Nutrients required for plant growth and reproduction can be positively charged, such as ammonium, potassium, boron, calcium, magnesium, and transition metals. These positively charged nutrients are poorly held in a negatively charged soil because binding mechanisms are non-selective, ionic charges which follow the rules of ion exchanged. For example, in soil, ions with low electropositive charges are rapidly and quickly displaced by ions with higher valence or larger electropositive charge. Thus, less than half of fertilizer added to agricultural soils is efficiently used to grow and reproduce crops. The modified amino acids can address a lack of positive charges in soils and the lack of selective chemical binding to control loss lower electropositively charged nutrients. Examples 1-3, provided herein, demonstrates importance of charge in the ability of the negative charges in the soil in complexing and retaining amino acids which have been modified to acquire a positive charge.

TABLE 1
Protonated Amino Acids

| | | | Nutrient |
|---|---|---|---|
| | Unmodified | Protonated | complexed |
| Amino Acid | Amino Acid | Amino Acid | and Bound |
| Arginine | C₆H₁₄N₄O₂ | C₆H₁₅N₄O₂ | Phosphate |
| Histidine | C₆H₉N₃O₂ | C₆H₁₀N₃O₂ | Phosphate |
| Lysine | C₆H₁₄N₂O₂ | C₆H₁₅N₂O₂ | Sulfate |
| Aspartic Acid | C₄H₇NO₄ | C₄H₈NO₄ | Nitrate |
| Glutamic Acid | C₅H₉NO₄ | C₅H₁₀NO₄ | Nitrate |
| Serine | C₃H₇NO₃ | C₃H₈NO₃ | Nitrate, Nitrite |
| Threonine | C₄H₉NO₃ | C₄H₁₀NO₃ | Nitrate, Nitrite |
| Asparagine | C₄H₈N₂O₃ | C₄H₉N₂O₃ | Sulfate |
| Glutamine | C₅H₁₀N₂O₃ | C₅H₁₁N₂O₃ | Sulfate |
| Cysteine | C₃H₇NO₂S | C₃H₈NO₂S | Nitrate, Nitrite |
| Selenocysteine | C₃H₇NO₂Se | C₃H₈NO₂Se | Nitrite, Nitrate |
| Glycine | C₂H₅NO₂ | C₂H₆NO₂ | Nitrate, Nitrite |
| Proline | C₅H₉NO₂ | C₅H₁₀NO₂ | Nitrate, Nitrite |
| Alanine | C₃H₇NO₂ | C₃H₈NO₂ | Potassium |
| Valine | C₅H₁₁NO₂ | C₅H₁₂NO₂ | Potassium |
| Isoleucine | C₆H₁₃NO₂ | C₆H₁₄NO₂ | Ammonium |
| Leucine | C₆H₁₃NO₂ | C₆H₁₅NO₂ | Ammonium |
| Methionine | C₅H₁₃NO₂ | C₅H₁₆NO₂ | Ammonium |
| Phenylalanine | C₉H₁₁NO₂ | C₉H₁₂NO₂ | Ammonium |
| Tyrosine | C₉H₁₁NO₃ | C₉H₁₂NO₃ | Nitrate |

TABLE 2
Ammonia Modified Amino Acids

| | | Ammonia | Nutrient |
|---|---|---|---|
| | Unmodified | Modified | complexed |
| Amino Acid | Amino Acid | Amino Acid | and Bound |
| Arginine | C₆H₁₄N₄O₂ | C₆H₁₅N₅O | Phosphate |
| Histidine | C₆H₉N₃O₂ | C₆H₁₀N₄O | Phosphate |
| Lysine | C₆H₁₄N₂O₂ | C₆H₁₅N₃O | Sulfate |
| Aspartic Acid | C₄H₇NO₄ | C₄H₈N₂O₃ | Nitrate |
| Glutamic Acid | C₅H₉NO₄ | C₅H₁₀N₂O₃ | Nitrate |
| Serine | C₃H₇NO₃ | C₃H₈N₂O₂ | Nitrate, Nitrite |
| Threonine | C₄H₉NO₃ | C₄H₁₀N₂O₂ | Nitrate, Nitrite |
| Asparagine | C₄H₈N₂O₃ | C₄H₉N₃O₂ | Sulfate |
| Glutamine | C₅H₁₀N₂O₃ | C₅H₁₁N₃O₂ | Sulfate |
| Cysteine | C₃H₇NO₂S | C₃H₈N₂OS | Nitrate, Nitrite |
| Selenocysteine | C₃H₇NO₂Se | C₃H₈N₂OSe | Nitrite, Nitrate |
| Glycine | C₂H₅NO₂ | C₂H₆N₂O | Nitrate, Nitrite |
| Proline | C₅H₉NO₂ | C₅H₁₀N₂O | Nitrate, Nitrite |
| Alanine | C₃H₇NO₂ | C₃H₈N₂O | Potassium |
| Valine | C₅H₁₁NO₂ | C₅H₁₂N₂O | Potassium |
| Isoleucine | C₆H₁₃NO₂ | C₆H₁₄N₂O | Ammonium |
| Leucine | C₆H₁₃NO₂ | C₆H₁₄N₂O | Ammonium |
| Methionine | C₅H₁₃NO₂ | C₅H₁₄N₂O | Ammonium |
| Phenylalanine | C₉H₁₁NO₂ | C₉H₁₂N₂O | Ammonium |
| Tyrosine | C₉H₁₁NO₃ | C₉H₁₂N₂O₂ | Nitrate |

TABLE 3
Guanidine Modified Amino Acids

| | | Guanidine | Nutrient |
|---|---|---|---|
| | Formula | Amino Acid | complexed |
| Amino Acid | Amino Acid | Formula | and Bound |
| Arginine | C₆H₁₄N₄O₂ | C₇H₁₉N₇O₂ | Phosphate |
| Histidine | C₆H₉N₃O₂ | C₇H₁₄N₆O₂ | Phosphate |
| Lysine | C₆H₁₄N₂O₂ | C₇H₁₉N₅O₂ | Sulfate |
| Aspartic Acid | C₄H₇NO₄ | C₅H₁₂N₄O₄ | Nitrate |
| Glutamic Acid | C₅H₉NO₄ | C₆H₁₄N₅O₄ | Nitrate |
| Serine | C₃H₇NO₃ | C₄H₁₂N₅O₃ | Nitrate, Nitrite |
| Threonine | C₄H₉NO₃ | C₅H₁₄N₄O₃ | Nitrate, Nitrite |
| Asparagine | C₄H₈N₂O₃ | C₅H₁₃N₅O₃ | Sulfate |
| Glutamine | C₅H₁₀N₂O₃ | C₆H₁₅N₅O₃ | Sulfate |
| Cysteine | C₃H₇NO₂S | C₄H₁₂N₄O₂S | Nitrate, Nitrite |
| Selenocysteine | C₃H₇NO₂Se | C₄H₁₂N₄O₂Se | Nitrite, Nitrate |
| Glycine | C₂H₅NO₂ | C₃H₁₀N₄O₂ | Nitrate, Nitrite |
| Proline | C₅H₉NO₂ | C₆H₁₄N₄O₂ | Nitrate, Nitrite |
| Alanine | C₃H₇NO₂ | C₄H₁₂N₄O₂ | Potassium |
| Valine | C₅H₁₁NO₂ | C₆H₁₆N₄O₂ | Potassium |
| Isoleucine | C₆H₁₃NO₂ | C₇H₁₈N₄O₂ | Ammonium |
| Leucine | C₆H₁₃NO₂ | C₇H₁₈N₄O₂ | Ammonium |
| Methionine | C₅H₁₃NO₂ | C₆H₁₈N₄O₂ | Ammonium |
| Phenylalanine | C₉H₁₁NO₂ | C₁₀H₁₆N₄O₂ | Ammonium |
| Tyrosine | C₉H₁₁NO₃ | C₁₀H₁₆N₄O₃ | Nitrate |

TABLE 4
Protonated Amino Acids

| | | | Nutrient |
|---|---|---|---|
| | Unmodified | Protonated | complexed |
| Amino Acid | Amino Acid | Amino Acid | and Bound |
| a-Amino-n-butyric acid | C₄H₁₀NO₂ | C₄H₁₁NO₂ | Nitrate |
| Norvaline | C₅H₁₁NO₂ | C₅H₁₂NO₂ | Nitrate |
| Norleucine | C₆H₁₃NO₂ | C₆H₁₄NO₂ | Nitrate |
| Alloisoleucine | C₆H₁₄NO₂ | C₇H₁₅NO₂ | Nitrate |
| t-leucine | C₆H₁₄NO₂ | C₆H₁₅NO₂ | Nitrate |
| a-Amino-n-heptanoic | C₇H₁₅NO₂ | C₇H₁₆NO₂ | |
| acid | | | |
| Proline | C₅H₁₀NO₂ | C₅H₁₁NO₂ | Nitrate |
| Pipecolic acid | C₆H₁₂NO₂ | C₆H₁₃NO₂ | Nitrate |
| a, β-diaminopropionic | C₅H₉N₂O₅ | C₅H₁₀N₂O₅ | Sulfate |
| acid | | | |
| a, γ-diaminobutyric acid | C₂₄H₂₉N₂O₂ | C₂₄H₃₀N₂0₂ | Sulfate |
| Ornithine | C₅H₁₂N₂O₂ | C₅H₁₃N₂0₂ | Sulfate |
| Allothreonine | C₄H₁₀NO₃ | C₄H₁₁NO₃ | Nitrate, Nitrite |
| Homocysteine | C₄H₉NO₂S | C₄H₁₀NO₂S | Nitrate, Nitrite |
| Homoserine | C₄H₁₀NO₃ | C₄H₁₁NO₃ | Nitrate, Nitrite |
| B-Alanine | C₃H₈NO₂ | C₃H₉NO₂ | Nitrate, Nitrite |
| B-Amino-n-butyric acid | C₄H₁₀NO₂ | C₄H₁₁NO₂ | Nitrate, Nitrite |
| B-aminoisobutyric acid | C₄H₁₀NO₂ | C₄H₁₁NO₂ | Nitrate, Nitrite |
| Isovaline | C₅H₁₂NO₂ | C₅H₁₃NO₂ | Nitrate, Nitrite |
| Sarcosine | C₃H₈NO₂ | C₃H₈NO₂ | Nitrate, Nitrite |
| N-ethyl glycine | C₅H₁₁NO₂ | C₅H₁₂NO₂ | Nitrate, Nitrite |
| N-propyl glycine | C₂₀H₂₂NO₄ | C₂₀H₂₃NO₄ | Ammonium |
| N-isopropyl glycine | C₁₁H₂₃N₂O₂ | C₁₁H₂₄N₂O₂ | Sulfate |
| N-methyl β-alanine | C₄H₁₀NO₂ | C₄H₁₁NO₂ | Nitrate |
| N-ethyl β-alanine | C₅H₁₂NO₂ | C₅H₁₃NO₂ | Nitrate |
| N-methyl alanine | C₄H₁₀NO₂ | C₄H₁₁NO₂ | Nitrate |
| N-ethyl alanine | C₅H₁₂NO₂ | C₅H₁₃NO₂ | Nitrate |
| Isoserine | C₃H₇NO₃ | C₃H₈NO₃ | Nitrate, Nitrite |
| a-hydroxy-γ- | C₄H₁₀NO₃ | C₄H₁₁NO₃ | Nitrate, Nitrite |
| aminobutyric acid | | | |

TABLE 5
Ammonia Modified Amino Acids

| | | Ammonia | Nutrient |
|---|---|---|---|
| | Unmodified | Modified | complexed |
| Amino Acid | Amino Acid | Amino Acid | and Bound |
| a-Amino-n-butyric acid | C₄H₁₀NO₂ | C₄H₁₁N₂O | Nitrate |
| Norvaline | C₅H₁₁NO₂ | C₅H₁₂N₂O | Nitrate |
| Norleucine | C₆H₁₃NO₂ | C₆H₁₄N₂O | Nitrate |
| Alloisoleucine | C₆H₁₄NO₂ | C₇H₁₅N₂O | Nitrate |
| t-leucine | C₆H₁₄NO₂ | C₆H₁₅N₂O | Nitrate |
| a-Amino-n-heptanoic | C₇H₁₅NO₂ | C₇H₁₆N₂O | |
| acid | | | |
| Proline | C₅H₁₀NO₂ | C₅H₁₁N₂O | Nitrate |
| Pipecolic acid | C₆H₁₂NO₂ | C₆H₁₃N₂O | Nitrate |
| a, β-diaminopropionic | C₅H₉N₂O₅ | C₅H₁₀N₃O₄ | Sulfate |
| acid | | | |
| a, γ-diaminobutyric acid | C₂₄H₂₉N₂O₂ | C₂₄H₃₀N₃O | Sulfate |
| Ornithine | C₅H₁₂N₂O₂ | C₅H₁₃N₃O | Sulfate |
| Allothreonine | C₄H₁₀NO₃ | C₄H₁₁N₂O₂ | Nitrate, Nitrite |
| Homocysteine | C₄H₉NO₂S | C₄H₁₀N₂OS | Nitrate, Nitrite |
| Homoserine | C₄H₁₀NO₃ | C₄H₁₁N₂O₂ | Nitrate, Nitrite |
| B-Alanine | C₃H₈NO₂ | C₃H₉N₂O | Nitrate, Nitrite |
| B-Amino-n-butyric acid | C₄H₁₀NO₂ | C₄H₁₁N₂O | Nitrate, Nitrite |
| B-aminoisobutyric acid | C₄H₁₀NO₂ | C₄H₁₁N₂O | Nitrate, Nitrite |
| Isovaline | C₅H₁₂NO₂ | C₅H₁₃N₂O | Nitrate, Nitrite |
| Sarcosine | C₃H₈NO₂ | C₃H₈N₂O | Nitrate, Nitrite |
| N-ethyl glycine | C₅H₁₁NO₂ | C₅H₁₂N₂O | Nitrate, Nitrite |
| N-propyl glycine | C₂₀H₂₂NO₄ | C₂₀H₂₃N₂O₃ | Ammonium |
| N-isopropyl glycine | C₁₁H₂₃N₂O₂ | C₁₁H₂₄N₃O | Sulfate |
| N-methyl β-alanine | C₄H₁₀NO₂ | C₄H₁₁N₂O | Nitrate |
| N-ethyl β-alanine | C₅H₁₂NO₂ | C₅H₁₃N₂O | Nitrate |
| N-methyl alanine | C₄H₁₀NO₂ | C₄H₁₁N₂O | Nitrate |
| N-ethyl alanine | C₅H₁₂NO₂ | C₅H₁₃N₂O | Nitrate |
| Isoserine | C₃H₇NO₃ | C₃H₈N₂O₂ | Nitrate, Nitrite |
| a-hydroxy-γ- | C₄H₁₀NO₃ | C₄H₁₁N₂O₂ | Nitrate, Nitrite |
| aminobutyric acid | | | |

Protonated Amino Acids

Protonated alanine is represented by formula (1).

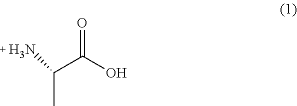

Protonated asparagine is represented by formula (2).

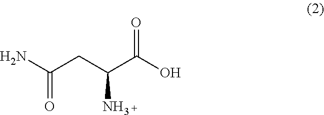

Protonated Aspartic acid is represented by formula (3).

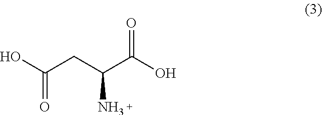

Protonated glutamic acid is represented by formula (4).

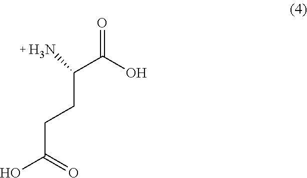

Protonated histidine is represented by formula (5).

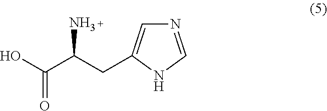

Protonated glycine is represented by formula (6).

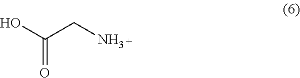

Protonated lysine is represented by formula (7).

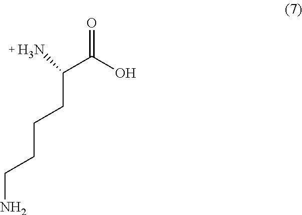

Protonated phenylalanine is represented by the formula (8).

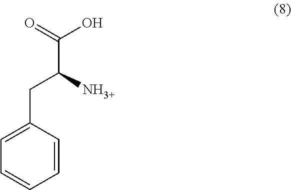 [SMILES: [NH3+][C@@H](Cc1ccccc1)C(=O)O]

Ammonia Modified Amino Acids

Ammonia modified valine is represented by the formula (9).

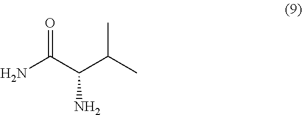

Ammonia modified alanine is represented by the formula (10).

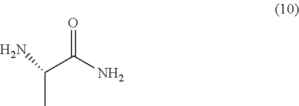

Ammonia modified glutamic acid is represented by formula (11).

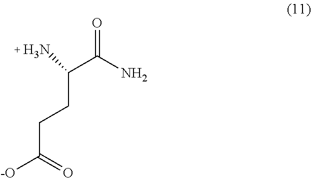

Ammonia modified glutamine is represented by formula (12).

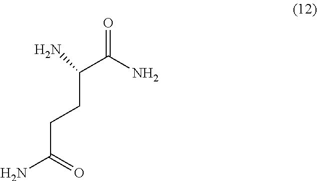

Ammonia modified tryptophan is represented by formula (13).

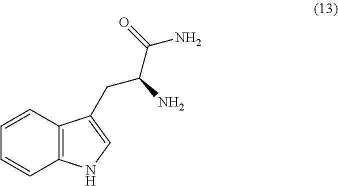 [SMILES: NC(=O)[C@@H](N)Cc1c[nH]c2ccccc12]

Ammonia modified methionine is represented by formula (14).

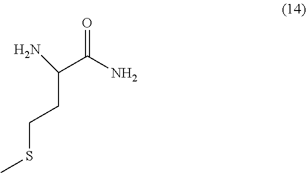

Guanidine modified leucine is represented by formula (15).

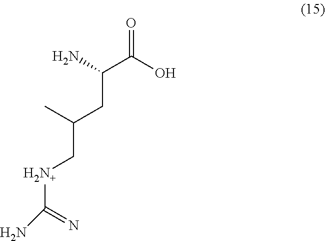

Guanidine modified isoleucine is represented by formula (16).

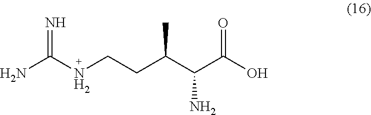

Unmodified Amino Acids

Unmodified amino acids can include arginine, histidine and lysine. Some amino acids are naturally positively charged and can be used in an unmodified state. For example, Arginine, Histidine and Lysine are naturally positively charged, and can be used in an unmodified state. However, these amino acids can also be used in a modified state as discussed herein. The modified stats discussed herein can increase positive charge, selectivity and nitrogen content of these amino acids.

Even if arginine, histidine and lysine are not modified, the method and timing of their application improves the efficiency of their retention in soils. The position of their placement in the soil as detailed in this invention and the timing of their placement in soil increase the efficiency of their utilization by plants.

Nitrogen and phosphorous application strategy of this invention targets fall application of biosynthesized nutrients with ion complexing and retention capabilities when silage is chisel plowed into the soil approximately 6-12 inches deep. Biosynthesized nutrients with ion complexing and retention capabilities can be applied by side dressing 3 inches deep during the spring when seeds are planted.

Since the biosynthesized nutrients with ion complexing and retention capabilities do not contain ammonium compounds, factors affecting ammonia gas losses do not apply.

Since the biosynthesized nutrients with ion complexing and retention capabilities do not contain nitrate/nitrite compounds, factors affecting nitrate/nitrite denitrification do not apply.

Since biosynthesized nutrients with ion complexing and retention capabilities are positively charged, they readily bond to the negatively charged soil particles; thus, factors affecting nutrient losses due to solubility in water are controlled and thus do not apply. Nor does nitrate runoff into waterway or groundwater nitrate contamination apply.

Since biosynthesized nutrients with ion complexing and retention capabilities contain specific ion complexing capabilities, microbial mineralization and/or mobilization of organic N and organic P in the soil into inorganic nitrates or phosphates is controlled; thus, nitrate or phosphate microbial mineralization or mobilization with subsequent loss by rain or irrigation, do not apply.

The benefits to farmers of applying modified or unmodified compositions of this invention are that fertilizer application labor is reduced to ⅓ or ¼ of the labor required to apply fertilizer 3 to 4 times a growing season. In addition to reduced labor is the reduced expense of operating and maintaining the fertilizer application equipment including fuel and other additives and labor. Finally, there is less wear and tear on fertilizer application equipment so that it will last longer and not prematurely require replacement.

If arginine, histidine and lysine are not modified, their solubility in soil/water solutions is very high. For example, at pH 7.0/8.0 and 25° C. solubility is: histidine 41.9 g/l, lysine 1,000 g/l, arginine 3,397 g/l. With the high solubility of lysine and arginine, they would be rapidly lost from soil due to rainfall or irrigation unless their positive charge is not increased by the modification techniques of this invention. Modification of lysine with a weak base to pH 10 would reduce its solubility by 89% to 110 g/l. Modification of arginine with a weak base to pH 10 would reduce its solubility by 93.3% to 228 g/l. High solubility of naturally occurring positively charged amino acids are subject to runoff, which may be problematic. However, positively charged amino acids are better retained than negatively charged nitrate, sulfate, and phosphate which are poorly retained in the negatively charged soils.

Method of Making Unmodified Amino Acids

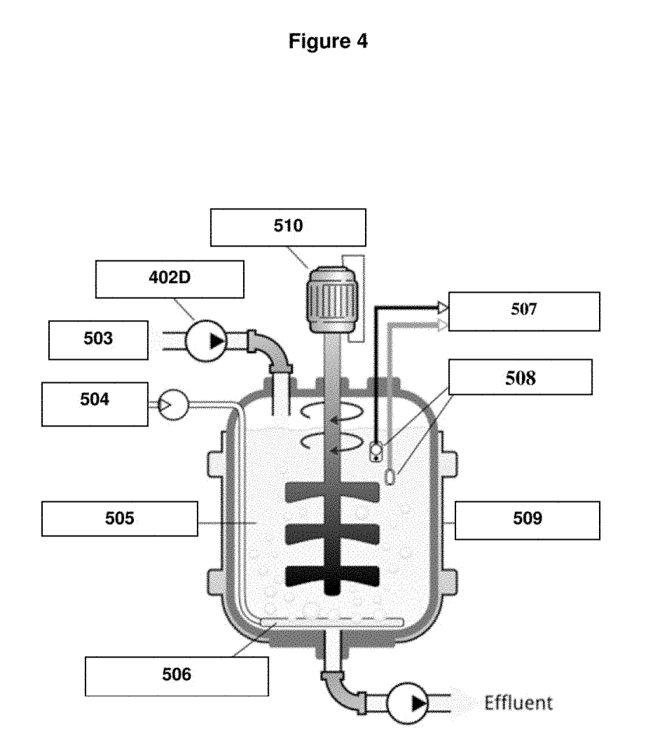

The preferred sources of raw material for the making of the product of this invention include high purity sources of carbohydrates including, cellulose, sugars and other simple and complex carbohydrates. Some preferred commercial sources of raw material for the synthesis of the products of this invention food scrapes from homes, institutions and restaurants, etc. and waste food products from farms, food stores, supermarkets and distribution networks in between these food outlets. Other preferred sources of raw materials for the making of the product of this invention include Waste Activated Sludge from wastewater treatment, animal manures and fowl manures. These raw materials provide nutrients to amino acid producing microorganisms that produce the unmodified amino acids. This method is described below in accordance with FIG. 4.

Methods of Making a Modified Amino Acid

A modified amino acid can be made by several methods depending on the functional group being added during modification.

Protonation of an amino acid follows the following reaction (1).

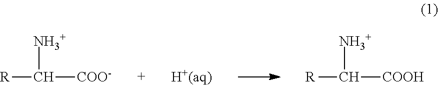

In the above reaction (1), a carboxylate group of the amino acid is protonated. The reaction is simply the transfer of the —H (positive ion) from the acid to the amine and the attraction of the positive and negative charges. The acid group becomes negative, and the amine nitrogen becomes positive because of the positive hydrogen ion. The carboxylate is then protonated to neutralize it.

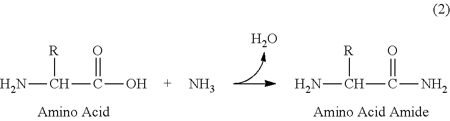

An amide modified amino acid follows the following reaction (2).
In the above reaction (2), a carboxyl group of the amino acid is modified with ammonia or an amine to form an amide.
An ester modified amino acid follows the following reaction (3).

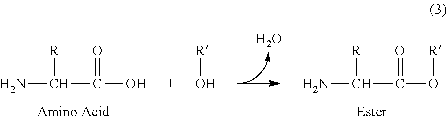

In the above reaction (3), a carboxyl group of the amino acid is reacted to form an ester. The ester can then be cationized.
A guanidine modified amino acid follows the following reaction (4).

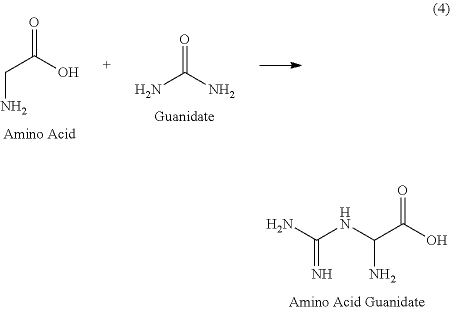

Although illustrated in isolation, the above reaction schemes (1) to (4) can be used in combination to make a modified amino acid, such as a modified amino acid that is both amine modified and protonated for example.

Through the use of the prior referenced amino acid modification procedures, amino acids and other raw materials can be made suitable for complexing and retaining nutrients in soils until needed by plants for their growth and reproduction. It has been discovered that the products of this invention can supply plant nutrient requirements directly into plants as amino acids; thus, they do not need to mineralize into elements to be nutritive to plants.

The first step in making the products of this invention is to determine the most technically and economically feasible source of raw materials to make the Specific Ion Complexing Agents of this invention. The lowest cost sources of raw materials are waste products from industry or municipal wastewater treatment. To be technically effective specific contaminate removal processes must be incorporated in pretreating waste raw materials so biosynthesis processes are not encumbered.

The second step in making the products of this invention is to utilize the optimum bio-reactor and to control supply of the critical amount of oxygen, nitrogen and other critical growth nutrients at the correct temperature for the correct time.

Method of Retaining Nutrients in Soil

The method may include analyzing a soil for anion charge content (Cation Exchange Capacity), organic content, nitrate concentration, ammonium concentration, pH, phosphate concentration, alkaline earth metal concentration, and/or transition metal concentration.

The method may include providing a composition including at least one modified amino acid based on analysis of the soil. The composition may include additional modified amino acids and/or unmodified amino acids as discussed herein. The unmodified and/or modified amino acids and amounts thereof for the composition may be selected based on one or more of the following factors, such as an amount of nitrate present in the soil to be complexed and retained, an amount of phosphate present in the soil to be complexed and retained, an amount of potassium present in the soil to be complexed and retained, an amount of sulfate present in the soil to be complexed and retained, an amount of alkaline earth metals present in the soil to be complexed and retained, and an amount of transition metals present in the soil to be complexed and retained.

Depending upon the amount of nutrient to be complexed and retained a specific dosage of Specific Ion Complexing Agent is applied. For example, to complex and retain about 120 pounds of nitrate in soil about 0.3 milliequivalents of a nitrate complexing amino acid is required per about 100 grams of soil.

The composition can be added or replenished in the soil as necessary. In some embodiments, the composition is added to the soil in the fall before plowing silage back into the soil, and/or the composition is added in the spring before or during seed planting.

Method of Waste Water Treatment to Recover Nutrients

Waste Water Treatment Plants (WWTP) typically produce solid wastes, such as biosolids, which have to be disposed of, for example, in a landfill or direct land application. The biosolids may include pathogens, heavy metals, vector attractants, and personal care products and pharmaceuticals (PCP&P). The biosolids are highly negatively charged organics which promote runoff and ground water contamination. Thus, the method of waste water treatment to recover nutrients provided herein removes valuable nutrients from the waste water stream, and reduces the amount of biosolids that are generated from WWTP. The methods provided herein can recover carbon, nitrogen, phosphorus, and potassium from WWTP. The recovered nutrients can be complexed with amino acids during a biosynthesis process. The complexed nutrients can be used as fertilizer. The methods provided herein can reduce fertilizer and/or synthetic fertilizer usage due to the reclaimed nutrients.

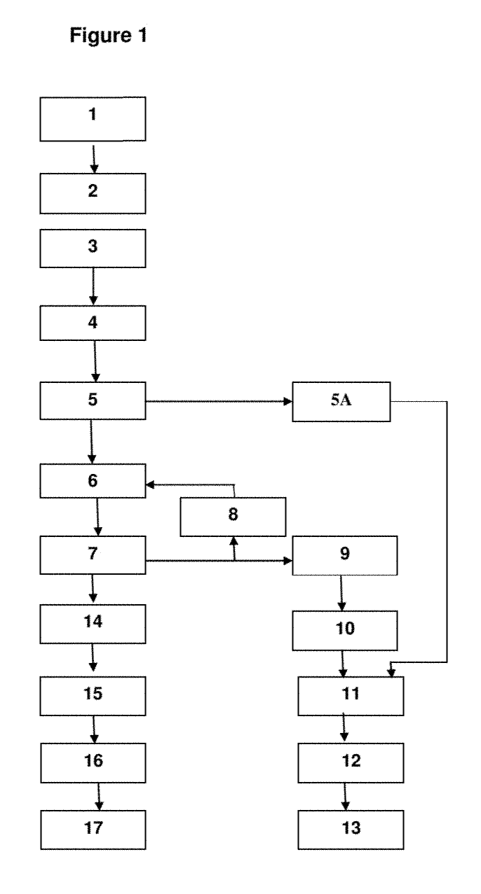

FIG. 1 depicts a process flow diagram of a conventional WWTP. At 1, wastewater enters the WWTP. At 2, large bulky items, such as rags and plastics are removed from the wastewater by a mechanical device or combination of devices, such as a bar screen. At 3, sand, cinders or other heavy solids are removed from the wastewater in a grit removal tank. At 4, fat, oil and grease, which floats on top of the wastewater is removed in primary influent channels. At 5, settleable solids which settle with velocity of about 0.5 ft/sec or lower are collected in primary sedimentation tanks. At 5A, primary sludge from 5 is directed to an anaerobic digestion process at 11. At 6, the wastewater from 5 is mixed with air and aerobic bacteria to remove organic carbon, nitrogen and phosphorus by biochemical oxidation in an activated sludge aerobic aeration tank. At 7, solids resulting from 6 are allowed to settle during secondary sedimentation. At 8, activated sludge from 7 is recycled again to 6 to remove additional organic carbon, nitrogen and phosphorus by biochemical oxidation. At 9, excess aerobic bacteria are removed from waste activated sludge that resulted from 7. The waste activated sludge contains about 0.5 wt % solids. At 10, the waste activated sludge is partially dewatered by a gravity belt thickener or similar process. The partially dewatered sludge contains about 3.5 wt % solids. The partially dewatered sludge is then directed to the anaerobic digestion process at 11. At 11, the anaerobic digestion process reduces total volatile solids (TVSs) by about 40 wt % or more, or about 40 wt % to about 50 wt % in a low oxygen environment and can generate biogas (low BTU gas CO₂ and methane) as a byproduct. The anaerobic digestion process lasts for about 20 to 30 days. The At 12, anaerobically digested sludge resulting from the anaerobic digestion process is dewatered to about 25 wt % to about 50 wt % dry solids in a screw press, centrifuge, drying bed, or a similar process. The partially dried solids resulting from 12 are disposed at 13 in a landfill, land applied, or the like.

Concurrently, the main wastewater flow from 7 proceeds to 14. At 14, nitrate or phosphate removal from the main wastewater flow can be achieved using a rotating bed Contractor (“RBC”) or a similar method. At 15, RBC solids are collected by tertiary clarifiers. At 16, the remaining waste water is disinfected. At 17, the waste water is discharged.

The process flow diagram shown in FIG. 1 can be applied to wastewater treatment flows ranging from 1000 GPD to over 1 billion GPD with many variations in between. The equipment/process steps can be consolidated in small plants or constructed in multiple trains in large plants. Piping and processing equipment dimensions are proportional to design capacity to achieve desired treatment results under varying flow conditions relative to plant design capacity.

Large wastewater treatment plants are constructed out of concrete whereas smaller treatment plants are constructed out of steel with various combinations of metallic and non-metallic materials of construction employed on a site-by-site basis.

The advantages of a conventional WWTP are that suspended solids are effectively reduced, biochemically degradable carbon is reduced, nitrates and phosphates are reduced and bacteria growth in the treated effluent is controlled.

Conventional WWTP are designed and operated to purify waste water within targeted limits. WWTP are not designed nor are they operated to reclaim valuable resources but rather in conventional WWTP approximately 50% or more of valuable resources are destroyed at the expense of valuable energy and other resources.

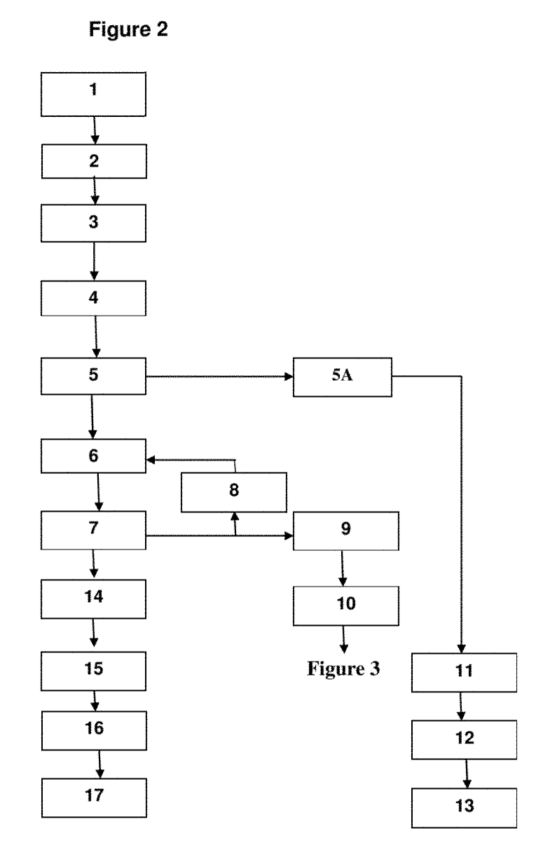

FIG. 2 depicts a process flow diagram of the conventional WWTP of FIG. 1 where steps 1 through 5 and 7 through 10 remain unchanged, and steps 11 through 13 apply only to primary sludge from 5A and not waste activated sludge from 10. Waste activated sludge from step 10 is directed to a biosynthesis process 400, which is depicted in FIG. 4. Unmodified or modified amino acids produced by the process 400 are returned to the conventional WWTP process at step 14 to scavenge additional nutrients from the waste water. Steps 15 through 17 also remain unchanged.

At 6, nitrogen and phosphorus concentration is further increased by adding additional sources of nitrogen, such as ammonia, and/or phosphates. Dissolved oxygen concentration can also be increased above about 2 mg/l and other essential nutrients can be added, as required, with an objective of increasing the nitrogen concentration of the activated bacteria to a range between 7 to 30%. Supplemental bacterial seed can also be added as necessary. As a result of the modifications at step 6, the solids at step 7 now contain higher amounts of nitrates and phosphates, as do the solids a steps 8 through 10.

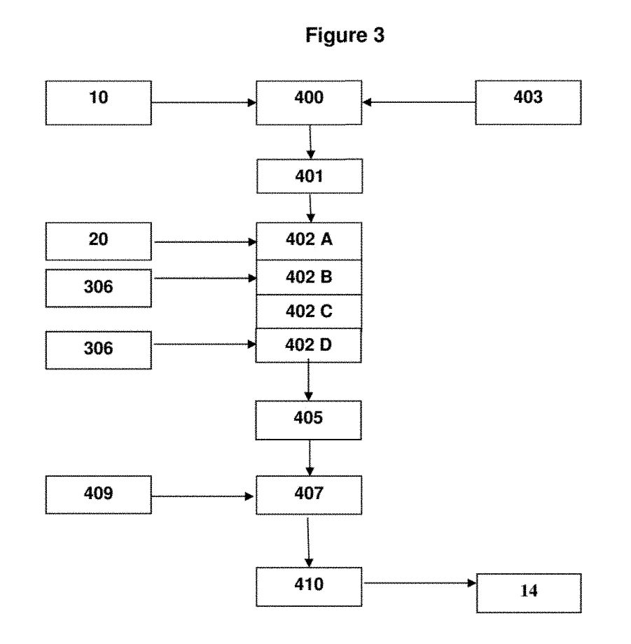

FIG. 3 depicts a process flow diagram for the biosynthesis process 400. The biological synthesis process 400 is supplied with partially dewatered activated sludge from 10 and a supply 403 of nitrogen and/or phosphorous and/or oxygen necessary to maintain a ratio of at least 5:1 nitrogen to phosphorous, and to maintain dissolved oxygen of above 2 mg/l. At 401, microbial solids are increased from about 3.5 wt % to about 5 wt % to about 10 wt % by a gravity thickening process. At 402, the solids of 401 are at least partially dewatered via a filter press, screw press, or the like to increase the solid concentration to about 25 wt % to 50 wt %. At 402A, free water containing dissolved salts is removed by compressed air blowing 20. At 402B, air dried solids from 402B contain about 50 wt % to 75 wt % water are rinsed with water from water source 306. At 402C, the rinsed solids are reacted with acid, such as 2N HCl and/or an inorganic acid and/or an organic acid, provided from acid source 1000. At 402D, the acid reacted solids of 402C which now contains a high amount of nutrients and low metal content are rinsed with water from water source 306.

At 405, a bioreactor 500 (see FIG. 4) receives rinsed solids from 402D and converts organically bound nitrogen or phosphates, e.g., bound in the aerobic bacteria of the WWTP, into inorganic nitrate or phosphate by breaking down the bacteria cell walls by any cell lysis method, such as high pressure dispersion of the bacteria against and through series of plates with small openings so that cell walls are destroyed partially or totally as desired. The freed nutrients are then used by the bioreactor (process described further below) to form unmodified amino acids.

Optionally, at 407, the unmodified amino acids can be reacted with modification agents from modification agent source 409 to produce modified amino acids. Modification agents can include one or more of protons, ammonia, guanidine, carbonate and alcohols.

At 410, the unmodified amino acids from 405 or the modified amino acids from 407 are dried. The drying can be direct or indirect heating. A tray dryer or another dryer that receives heated gas can be utilized. The dried unmodified or modified amino acids may have a moisture content of about 10 wt % or less. The dried unmodified or modified amino acids can then be stored for delivery to end users, and/or packaged in super-sacks containing up to 2000 pounds (lbs.), 1-2 cubic foot packages containing about 35 to about 70 lbs, or packages containing about 5 to 10 lbs.

At 412, at least a portion of the dried modified or unmodified amino acids can be returned to step 14 in the WWTP process to aide in nitrate and/or phosphate recovery at step 14.

Returning to FIG. 2, at step 15, the modified or unmodified amino acids, now charged with nutrients such as nitrates and/or phosphates at step 14, are removed during clarification.

The advantages the processes described in FIGS. 2 and 3 over conventional WWTP is reduction in waste solids, aerobic bacteria presence in solids that are generated is low, and purchased energy costs are lower than conventional WWTP costs while reclaimed carbon is increased about 33% to about 80% over conventional WWTP. The modified WWTP as described in FIGS. 2 and 3, equal or exceed the quality of wastewater treatment achieved at a substantially lower cost, valuable resources including carbon, nutrients and essential minerals and salts are reclaimed, significant reduction in carbon dioxide achieved, but also SICA are biologically synthesized allowing cost effective control of nutrient runoff from agricultural and non-agricultural sites when the products of this invention are incorporated on same.

FIG. 4 depicts the bioreactor 500 that may be used in the process 400. Alternatively, the bioreactor can be used independently of process 400 by providing nutrient sources to produce unmodified or modified amino acids. As descripted herein, the bioreactor 500 will utilized nutrient sources as discussed at step 402D. The bioreactor 500 includes a tank 505. The tank 505 includes an aerator 506 disposed therein and an agitator system 410 disposed therein. A medium supply 503 and air/gas supply 404 are coupled to the tank 505 for introducing materials into the tank 505. The bioreactor 505 may include a system monitor 407 and sensor probes 408 disposed in the tank for monitoring processes, temperature and the like. The bioreactor may include a jacket 409, such as a cooling or heating jacket, for controlling temperature of the tank interior.

In operation, the solids from step 402D are supplied to the tank 505. The medium being supplied from supply 503 contains 5 to 20% microbiological organisms and 60 to 90% volatile solids. Besides microorganisms, the medium contains organic and inorganic particles and extracellular polymers composed mostly of carbohydrates. The extracellular polymers comprises 15% to 20% of the volatile total solids on a dry weight basis. Protozoa and other higher life forms, including flagellates, amoebae, free-swimming and attached ciliates, rotifers and higher invertebrates, constitute approximately 5% of the medium microbes. Approximately 95% of the microbes include genera such as Pseudomonas, Achromobacter, Flavobacterim, Alcaligenes, Arthrobacter, Citromonas, and Zoogloea. Nutritional requirements for bacterial growth and reproduction include carbon, nitrogen and phosphorous in ratio of 100:5:1, such as the nutrients supplied from the solids of 402D. The typical composition of bacterial cells in the solids of 402D is:

TABLE 6
Composition of Bacterial Cells

| | | | |
|---|---|---|---|
| Carbon | 50 | Potassium | 1 |
| Oxygen | 20 | Sodium | 1 |
| Nitrogen | 14 | Calcium | 0.5 |
| Hydrogen | 8 | Magnesium | 0.5 |
| Phosphorous | 3 | Iron | 0.2 |
| Sulfur | 1 | All other elements | 0.3 |

For example, a typical analysis of a Waste Activated Sludge medium fed to bioreactor 505 would contain about 3.5 wt % total solids of which about 70 wt % is organic. The organic composition is about 7 wt % organic nitrogen, about 0.2 wt % ammonia, and about 2 wt % phosphorous. Microbe growth in the medium is nutrient and oxygen limited. As discussed in process 400, additionally nutrients can be provided to further stimulate microbe growth and prevent microbe nutrient deficiency.

The microbiological organisms of the medium include various strains of amino acid-producing bacteria, such as L-arginine-producing strain (ATCC 21659) Canananine resistant) obtained from Corynebacterium Glutamicum (synonym of Micrococcus Glutamicus) ATCC 13032, or L-arginine-producing strain (Canavanine resistant) of Corynebacterium glutamicum ATCC 21831. Laboratory tests demonstrated growth of L-Arginine of 11.9% in 72 hours. This effectively increased the nitrogen content of the microbial mass by 170.1%. The resulting nitrogen concentration in the microbial mass was 18.9 wt % which was 26% above the target of 15 wt % nitrogen concentration.

Variation of the amino acids cultured in the microbial mass provides the binding sites for formation of Specific Ion Complexing Agents for various anions and cations. Nitrogen is contained in the single cell proteins in the form of amino acids. Depending on the identity of the amino acid-producing bacteria in the microbial mass, specific amino acids are produced and the nitrogen concentration of the microbial mass can vary. For example, amino acids in the table below have nitrogen content that varies from 13.7 to 32.0 percent based on the identity of the amino acid.

TABLE 7
Nitrogen Content in Amino Acids.

| | Molecular | | Nitrogen |
|---|---|---|---|
| Amino Acid | Weight | Formula | Percentage |
| Asparagine | 123.12 | C₄H₈N₂O₃ | 21.2 |
| Glutamine | 146.15 | C₅H₁₀N₂O₃ | 19.2 |
| Histidine | 155.16 | C₆H₉N₃O₂ | 27.0 |
| Lysine | 146.19 | C₆H₁₄N₂O₂ | 19.2 |
| Arginine | 174.2 | C₆H₁₄N₄O₂ | 32.0 |
| Tryptophan | 204.23 | C₁₁H₁₂N₂O₂ | 13.7 |
| Glycine | 75.07 | C₂HfNO₂ | 18.7 |

Thus nitrogen concentration in the final product, i.e., the unmodified or modified amino acids that are formed, is controlled by the type of amino acid that is being formed.

### Example 1—Protonation of Carboxylate

89.09 grams of alanine was dissolved in a 3 liter reactor containing 991 ml of deionized water with mixing at 30 rpm. After 15 minutes, 1.0 molar HCl as a 31.5% solution was added dropwise to reach pH of 4.05. When pH had stabilized, the solution was extracted with 95% ethyl alcohol. The alanine water alcohol mass was dried to 103° C.

After drying, 890.9 mg of the modified alanine (i.e., protonated alanine) was added to 100 ml of deionized water to form a modified alanine solution. The electro-potential of the modified alanine water mixture was measured with an electro-potential meter against a hydrogen electrode. The modified alanine measure positive 177 eV. 890.9 mg sample of unmodified alanine was added to 100 ml of deionized water. The electro-potential of the unmodified alanine was negative 4 eV.

The modified alanine solution was added to 100 ml burette with 9 mm diameter at the rate of 1.5 ml/min. The column was filled with 89 ml of WS A201 (WaterScience, Inc., Peoria, Ill.) which has an anion exchange capacity of 2 EG Kg. To the burette was added 100 ml deionized water to displace the modified alanine. 45.1 mg of the modified alanine passed through the column by the displacement water. 845.8 mg of modified alanine was complexed and retained by the WS A 201. The 100 ml of deionized water containing 890.9 mg of unmodified alanine was added to another column containing fresh WS A 201. To the burette was also added 100 ml of deionized water to displace the unmodified alanine solution from the column. 37.3 mg of unmodified alanine was complexed and retained by the WS A 201. 853.6 mg of unmodified alanine passed through the column. 94.9% of the protonated alanine was converted to cationic charge.

### Example 2—Ammonia Reaction with Carboxyl Group

147.13 grams of glutamic acid was dissolved in a 3 liter reactor containing 853 ml of deionized water with mixing at 30 rpm. After 15 minutes, 18 grams of ammonia as a 28% solution was added dropwise to the reactor. After 15 minutes, the solution was extracted with 95% ethyl alcohol. The glutamic acid water alcohol mass was dried to 103° C.

After drying, 1471 mg of the modified glutamic acid was added to 100 ml of deionized water to form a modified glutamic acid solution. The electro-potential of the modified glutamic acid water mixture was measured with an electro-potential meter against a Hydrogen electrode. The modified glutamic acid measured positive 167 eV. 890.9 mg sample of unmodified glutamic acid was added to 100 ml of deionized water. The electro-potential of the unmodified glutamic acid was negative 9 eV.

The modified glutamic acid solution was added to 100 ml burette with 9 mm diameter at the rate of 1.5 ml/min. The column was filled with 89 ml of WS A 201 which has an anion exchange capacity of 2 EG Kg. To the burette was added 100 ml deionized water to displace the modified glutamic acid. 98 mg of the modified glutamic acid passed through the column by the displacement water. 1373 mg of modified glutamic acid was complexed and retained by the WS A 201. The 100 ml of deionized water containing 1471 mg of unmodified glutamic acid was added to another column containing fresh WS A 201. To the burette column was also added 100 ml of deionized water to displace the unmodified glutamic acid solution from the column. 79 mg of the unmodified glutamic acid was complexed and retained by the WS A 201. 1392 mg of unmodified glutamic acid passed through the column. 93.3% of the ammonia modified glutamic acid was converted to cationic charge.

### Example 3—Guanidine Reaction with Side Chain Group

131.18 grams of leucine was dissolved in a 3 liter reactor containing 869 ml of deionized water with mixing at 30 rpm. After 15 minutes, 59 grams of guanidine as a 99% solution was added dropwise to the reactor. After 15 minutes, the solution was extracted with 95% ethyl alcohol. The guanidine leucine water alcohol mass was dried to 103° C.

After drying, 1902 mg of the modified leucine was added to 100 ml of deionized water to form a modified leucine solution. The electro-potential of the modified leucine water mixture was measured with an electro-potential meter against a Hydrogen electrode. The modified leucine measured positive 197 eV. 1312 mg sample of unmodified leucine was added to 100 ml of deionized water. The electro-potential of the unmodified leucine was negative 18 eV.

The 1% modified leucine acid solution was added to 100 ml burette with 9 mm diameter at the rate of 1.5 ml/min. The column was filled with 89 ml of WS A 201 which has an anion exchange capacity of 2 EG Kg. To the burette was added 100 ml deionized water to displace the modified leucine. 66 mg of the modified leucine passed through the column by the displacement water. 1246 mg of modified leucine was complexed and retained by the WS A 201. The 100 ml of deionized water containing 1312 mg of unmodified leucine was added to another column containing fresh WS A 201. To the burette was also added 100 ml of deionized water to displace the unmodified leucine solution from the column. 41 mg of the unmodified leucine was complexed and retained by the WS A 201. 1271 mg of unmodified leucine passed through the column. 95% of the guanidine modified leucine acid was converted to cationic charge.

### Example 4—Biosynthesis of Arginine

20 ml Batches of an aqueous medium containing I 0% Waste Activated Sludge, 6% ammonium sulfate, 0.1% potassium dihydrogen phosphate, (2.4% total nitrogen) were sterilized in respective 500 ml shaking flasks and adjusted to pH 7 with 5% separately sterilized calcium carbonate.

Inocula of B. flavum AJ 340 I prepared on bouillon agar slants were added to each flask which was thereafter held at 31° C. with aeration and agitation for 72 hours. The combined broth contained 2.5 g/dl arginine and was centrifuged to remove the cells. One liter of the supernatant liquid was passed over a column packed with an ion exchange resin (Amberlite C-50, NH4 type), and the arginine adsorbed by the resin was eluted with 2-N ammonium hydroxide solution. The eluate was partly evaporated to precipitate crude, crystalline arginine which, when dried, weighed 17.8.

The following examples are given to illustrate the invention in greater detail. Unless otherwise indicated, all parts, percents, ratios and the like are by weight.

### Example 5—Biosynthesis of Lysine

Pseudomonas brevis (ATCC-21941) as a hydrocarbon assimilating and L-lysine producing microorganism was cultured on a bouillon agar slant at 33° C. for 24 hours, and then was used to inoculate the following seed culture medium and was then cultured at 33° C. The composition of the seed culture medium was as follows: Waste Activated Sludge 5 g/1, 75% HaPO₄ 12 ml/L, (NH₄HSO₄ 6 g/L, NaCl I g/L, MgSO₄.7H₂O 0.2 g/L, CaCl₂.2H₂O 0.1 g/L, FeSO₄.7H₂O 0.1 g/L, ZnSO₄.7H₂O 0.03 g/L, and MnSO₄.4H₂O 0.0002 g/L. The pH was adjusted to about 7.0 with KOH. This seed medium was also employed in Example. After 24 hours, 1 ml of the above described seed culture (inoculum ratio ca. 3%) was used to inoculate 30 ml of a fermentation medium in shaking-flasks which was sterilized at 120° C. for 30 min. and cultured with shaking. at 33° C. The composition of the fermentation medium was as follows:

TABLE 8
Composition of Medium

| Waste Activated Sludge | 11.6% w/v |
|---|---|
| K₂HPO₄ | 0.1% w/v |
| KH₂PO₄ | 0.1% w/v |
| (NH₄)₂SO₄, | 3.5% w/v |
| pH | 7.0 |

After 24 hours from the beginning of the cultivation, a platinum loopful amount of each of the hydrocarbon non-assimilating bacteria as shown in Table I below was used to inoculate the culture to provide a mixed culture and the culturing was continued for 9 days. A control culture was also conducted using only the Pseudomonas brevis for the purposes of comparison. The concentration of L-lysine (as the hydrochloride) produced in the broth of each of the fermentations was measured at 7 and 9 days using a microbio assay method in which an L-lysine auxotroph of Escherichia coli or Leuconostoc mesentroides is used. The results obtained are shown in Table 9.

TABLE 9

| | L-Lysine (HCI) Accumulated | |
|---|---|---|
| | 7 Days | 9 Days |
| Strains of Mixed Bacteria | mg/ml | mg/ml |
| Pseudumonas brevis Control* | 20.7 | 27.0 |
| Pseudomonas brevis plus | | |
| Bacillus megatherium IAM-1030 | 25.5 | 30.6 |
| Bacillus subtilis IAM-I145 | 24.6 | 30.6 |
| Bacillus po/ymyxa JAM-I I 89 | 31.0 | 33.2 |
| Microbacterium j/avum ATCC-10340 | 31.3 | 35.3 |
| Microbacterium lacticum JAM-1640 | 28.7 | 30.9 |
| Micrococcus candidus ATCC-14852 | 33.4 | 33.9 |
| Brevibacterium ammoniagenes JAM-1641 | 29.9 | 32.2 |
| Aeromonas formicans ATCC-1 3 13 7 | 36.7 | 36.6 |
| Aerobacter clocae JAM-I020 | 29.6 | 30.5 |
| Escherichia coli K-12 | 37.2 | 38.7 |
| Corynebacterium Jaciens IAM-1079 | 33.4 | 28.0 |
| Corynebacterium rathayi ATCC- I 3659 | 29.2 | 32.3 |
| Proteus vulgaris IAM-1025 | 33.5 | 31.4. |

*Single culture using Pseudomonas brevis only.

As shown by the results in Table 9 above, the production of L-lysine was greatly improved when a mixed culture of the hydrocarbon assimilating microorganism and the hydrocarbon non-assimilating microorganism was employed.

The time required to reach the maximum yield was also 7 to 9 days in the case of a mixed culture, while it was 10 to 11 days in the case of a single culture. Moreover, it was found that the microbial cells were easily removed by filtration or centrifuging after heating the broth at 80° to 100° C.

It was also found that the capability for L-lysine production of the mixed microorganisms listed above was comparable to prior art when they were cultured in a medium containing Waste Activated Solids as carbon source. Thus, it was supposed that the advantageous effect of the mixed culture method was not due to the production of L-lysine by the hydrocarbon non-assimilating microorganisms, but, for instance, to a stimulating effect by Waste Activated Solids to the microorganisms.

### Example 6—Method of Nutrient Retention in Soil

The composition used in the study below includes guanidine modified leucine present in an amount of about 70 wt %, guanidine modified isoleucine present in an amount of about 10 wt %, proton modified asparagine present in an amount of about 10 wt %, ammonia modified valine present in an amount of about 5 wt %, and ammonia modified alanine present in an amount of about 5 wt %. The composition was added to the experimental example at a rate of about 1,200 lbs per acre.

Two 7 acre plots were selected from a 69 acre farm site. A 7 acre control site was located on the south side of the farm. The soil was identified as Ipava Silt Loom. A 7 acre experimental site was located on the north side of the farm. The soil on that site was identified as Clarksdale Silt Loom.

Historically, the southern control site had higher yields than the northern experimental site. In the prior year the control site yielded 257 bushels of corn per acre whereas the experimental site yielded 251 bushels of corn. The control site historically yielded 2.4% higher than the experimental. All yields were determined by continuous monitoring during harvest.

Analyses of the chemical composition of the soils at both 7 acre sites were performed prior to the growing season. Organic nitrogen, ammonium nitrogen and nitrate nitrogen in the top 12 inches of the soil of the control site were collectively 51.8% higher than the top 12 inches of the soil of the experimental site. Organic nitrogen, ammonium nitrogen and nitrate nitrogen in the top 13-24 inches of the soil of the control site were collectively 49.7% higher than the top 13-24 inches of the soil of the experimental site. These chemical analyses explain why the Southern control site historically outperforms the Northern experimental site.

TABLE 10
Beginning soil analyses; Control versus Experimental

| | | | | | Control |
|---|---|---|---|---|---|
| | Soil | Control | Soil | | Increase |
| | Sample | Site | Sample | Experimental | Over |
| Parameter | Location | Mg/l | Location | Site Mg/l | Experimental |
| Organic N | C-12 | 1,393.6 | E-12 | 777.2 | 79.3% |
| Ammonium N | | 616.4 | | 562.8 | 9.5% |
| Nitrate N | | 44.8 | | 13.4 | 234.0% |
| Organic N | C-24 | 991.6 | E-24 | 509.2 | 94.7% |
| Ammonium N | | 616.4 | | 562.8 | 9.5% |
| Nitrate N | | 6.0 | | 5.9 | 1.7% |

12 inch and 13-24 inch soil samples were collected from four equidistant sampling points after all fertilizers had been applied to both the northern and southern sites.

TABLE 11
Fertilizer addition summary showing 25.7% more nitrogen added to
control site

| | | | |
|---|---|---|---|
| Fertilizer | Quantity | Application | Addition |
| Added | Added | Method | Date |

Experimental

| | | | | |
|---|---|---|---|---|
| | UAN + Agrotain | 50 # N | Banded | Apr. 18, 2016 |
| | Ultra | | | |
| | UAN | 40 # N | Broadcast | Apr. 19, 2016 |
| | Invention | 77 # N | Broadcast | Apr. 16, 2016 |
| Total | | 167 # N | | |

Control

| | | | | |
|---|---|---|---|---|
| | UAN + Agrotain | 50 # N | Banded | Apr. 18, 2016 |
| | Ultra | | | |
| | UAN | 40 # N | Broadcast | Apr. 19, 2016 |
| | UAN | 120 # N | Banded | Jun. 8, 2016 |
| Total | | 210 # N | | |

Growing Season Yields

The 7 acre Control site had yields averaging 238 bushels per acre as determined by continuous yield monitoring during harvesting. The 7 acre Experimental site had yields averaging 236 bushels per acre as determined by continuous yield monitoring during harvesting.

Historically the control site yields 2.4% higher than the experimental site due to the much higher nutrient concentration in the control site soil. When the experimental site yield is corrected for the higher starting nutrient concentration and the historical advantage of the superior control soil, the handicapped adjusted yield for the experimental site is 241.7 bushels.

Complexing and Retaining Major Nutrients in Soil

Review of the below table shows the vastly improved capabilities of the invention to complex and retain major nutrients in the soil for use in succeeding growing seasons.

TABLE 12
Ending Soil Analyses for the 12″ deep and 24″ deep samples combined
organic nitrogen, ammonium nitrogen and nitrate nitrogen.

| | | | | % Difference |
|---|---|---|---|---|
| Element/ | Begin | End | Pounds | Experimental |
| Compound | Apr. 5, 2016 | Sep. 21, 2016 | Difference | over Control |
| Total Nitrogen | | | | |
| ppm | | | | |
| Control 12 Inches | 2,054.8 | 1,876.0 | −178.0 | −8.7 |
| Control 24 Inches | 1,614.0 | 999.6 | −614.4 | −38.1 |
| Total | 3668.8 | 2875.6 | −792.4 | −21.6 |
| Experiment | 1,353.4 | 1,476.1 | +122.7 | +9.1 |
| 12 Inches | | | | |
| Experiment | 1077.9 | 919.2 | −158.7 | −14.7 |
| 24 Inches | | | | |
| Total | 2,431.3 | 2,395.3 | −36.0 | −1.5 |

Table 12 shows the considerable starting advantage that the control site had in nitrogen 3668.8 pounds/acre versus the experimental site nitrogen of 2,431.3 pounds/acre. The table also shows that the control site consumed 792.4 pounds of nitrogen per acre whereas the experimental site consumed 36 pounds of nitrogen per acre. This was dramatically reflected in the nitrogen analyses in the tile drain water where the nitrate concentration of the experimental site was about 60 to 70% lower than the control site. The grain leaving both sites contained approximately 200 pounds of nitrogen. Thus, Nitrogen Utilization Efficiency (NUE) for the control site was 25.2% whereas the NUE for the experimental site was a positive 82.0%.

Complexing and Retaining Major and Minor Elements and Nutrients in Soil

Review of the Table 13 below shows the vastly improved capabilities of the invention to complex and retain major and minor elements in the soil for use in succeeding growing seasons.

TABLE 13
Ending Soil Analyses for the 7″ deep samples for major and
minor elements.

| Element/ | Beginning 7″ ppm | Ending 7″ ppm | % Difference |
|---|---|---|---|
| Compound | Apr. 5, 2016 | Feb. 24, 2017 | Begin vs. End |
| Control | | | |
| Phosphorous Bray P1 | 87 | 87 | 0.0 |
| Phosphorous Bray P2 | 107 | 140 | 30.8 |
| Potassium | 229 | 137 | −40.2 |
| Calcium | 3302 | 2401 | −27.3 |
| Magnesium | 426 | 490 | 15.0 |
| Organic Matter | 4.6% | 3.4% | −26.1 |
| CEC meq/100 grams | 20.6 | 18.0 | −12.6 |
| Experimental | | | |
| Phosphorous Bray P1 | 60 | 83 | 38.3 |
| Phosphorous Bray P2 | 115 | 128 | 11.3 |
| Potassium | 131 | 144 | 9.9 |
| Calcium | 2525 | 2427 | −3.9 |
| Magnesium | 335 | 523 | 56.1 |
| Organic Matter % | 3.0 | 3.0 | 0.0 |
| CEC meq/100 grams | 17.1 | 19.3 | 12.9 |

Table 13 again shows the considerable starting advantage that the control site had in major and minor elements. With the exception of phosphorous and magnesium, the control site lost 12.6 to 40.2% of the major and minor elements due to tile drain water losses. Review of the experimental losses all of the major and minor elements showed an increased retention in the soil except for 3.9% of the beginning calcium.

Because of the high NUE and the high retention of the major and minor nutrients and elements the amount of invention used for the subsequent growing season was be 50% less than that required using synthetic fertilizers. It is not only were high yields continued and environmental pollution abated but fertilizer and other chemical usage were half that of traditional chemicals.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition comprising:
at least one modified amino acid.

2. The composition of claim 1, wherein the at least one modified amino acid includes a protonated amino acid, an ammonia modified amino acid, or a guanidine functionalized amino acid.

3. The composition of claim 2, where the protonated amino acid is at least one selected from the following compounds:

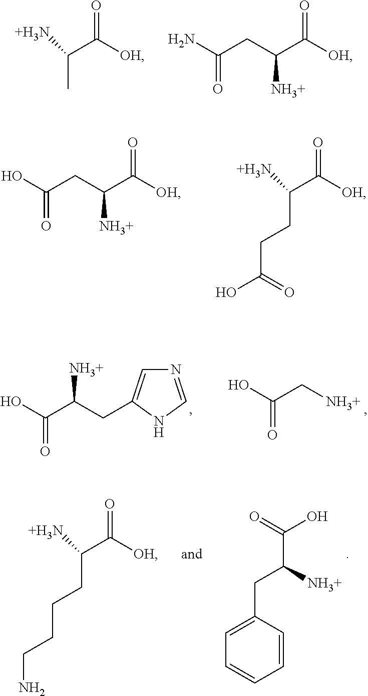

4. The composition of claim 2, where the ammonia modified amino acid is at least one selected from the following compounds:

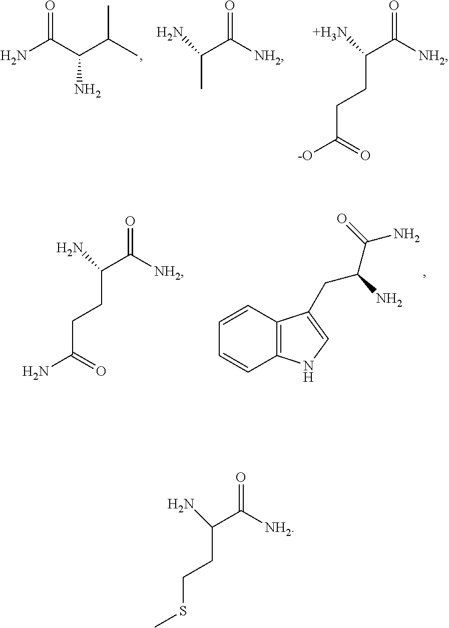

5. The composition of claim 2, where the guanidine functionalized amino acid is at least one selected from the following compounds:

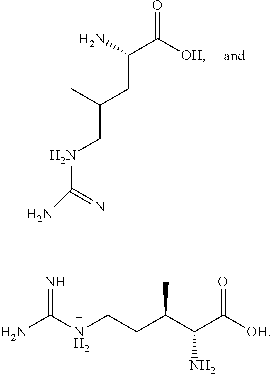

6. The composition of claim 1, further comprising:
at least one unmodified amino acid.

7. The composition of claim 6, where the at least one unmodified amino acid is selected from the group consisting of arginine, lysine, and histidine.

8. The composition of claim 6, comprising: histidine, protonated alanine, lysine, and protonated phenylalanine.

9. The composition of claim 6, comprising: histidine, ammonia modified glutamic acid, ammonia modified valine, ammonia modified tryptophan, and ammonia modified methionine.

10. The composition of claim 6, comprising: guanidine modified leucine, guanidine modified isoleucine, guanidine modified asparagine, and guanidine modified valine.

11. The composition of claim 6, wherein the at least one unmodified amino acid is selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

12. The composition of claim 6, wherein the at least one unmodified amino acid is selected from the group consisting of a-amino-n-butyric acid, norvaline, norleucine, alloisoleucine, t-leucine, a-amino-n-heptanoic acid, proline, pipecolic acid, a, β-diaminopropionic acid, a, γ-diaminobutyric acid, ornithine, allothreonine, homocysteine, homoserine, B-alanine, B-amino-n-butyric acid, B-aminoisobutyric acid, isovaline, sarcosine, N-ethyl glycine, N-propyl glycine, N-isopropyl glycine, N-methyl β-alanine, N-ethyl β-alanine, N-methyl alanine, N-ethyl alanine, isoserine, a-hydroxy-γ-aminobutyric acid, and mixtures thereof.

13. The composition of claim 2, wherein the protonated amino acid is a protonated form of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

14. The composition of claim 2, wherein the ammonia modified amino acid is an ammonia modified form of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

15. The composition of claim 2, wherein the guanidine modified amino acid is a guanidine modified form of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and mixtures thereof.

16. The composition of claim 2, wherein the protonated amino acid is a protonated form of a-amino-n-butyric acid, norvaline, norleucine, alloisoleucine, t-leucine, a-amino-n-heptanoic acid, proline, pipecolic acid, a, β-diaminopropionic acid, a, γ-diaminobutyric acid, ornithine, allothreonine, homocysteine, homoserine, B-alanine, B-amino-n-butyric acid, B-aminoisobutyric acid, isovaline, sarcosine, N-ethyl glycine, N-propyl glycine, N-isopropyl glycine, N-methyl β-alanine, N-ethyl β-alanine, N-methyl alanine, N-ethyl alanine, isoserine, a-hydroxy-γ-aminobutyric acid, and mixtures thereof.

17. The composition of claim 2, wherein the ammonia modified amino acid is an ammonia modified form of a-amino-n-butyric acid, norvaline, norleucine, alloisoleucine, t-leucine, a-amino-n-heptanoic acid, proline, pipecolic acid, a, β-diaminopropionic acid, a, γ-diaminobutyric acid, ornithine, allothreonine, homocysteine, homoserine, B-alanine, B-amino-n-butyric acid, B-aminoisobutyric acid, isovaline, sarcosine, N-ethyl glycine, N-propyl glycine, N-isopropyl glycine, N-methyl β-alanine, N-ethyl β-alanine, N-methyl alanine, N-ethyl alanine, isoserine, a-hydroxy-γ-aminobutyric acid, and mixtures thereof.
